(19) United States
(12) Patent Application Publication
(10) Pub. No.: US 2012/0190924 A1
(43) Pub. Date: Jul. 26, 2012

(54) Flexible Tubular Interlocking Structure for a Handheld Endoscope

(76) Inventor: Hsiang Te Tseng, New Taipei City (TW)

(21) Appl. No.: 13/100,268

(22) Filed: May 3, 2011

(30) Foreign Application Priority Data
Jan. 21, 2011 (TW) 100201401

(51) Int. Cl. A61B 1/008

(52) U.S. Cl. 600/127; 600/141

(57) ABSTRACT

A flexible interlocking structure for a handheld endoscope includes a flexible rod having an interlocking unit connected to its one end. The interlocking unit includes front, middle and rear joints each having a hollow portion. Each joint is formed with at least two guiding holes, locking tabs and locking notches. The engagement between the locking tabs and the locking notches of the adjacent joints constitutes the interlocking unit. Both ends of the flexible rod are connected to a handle portion and an inspection module respectively. A transmission cable and driving wires are disposed through the hollow portions and the guiding holes in such a manner that they are located between the inspection module and the handle portion. By operating a steering device of the handle portion, the driving wires pulls the front joints to cause the interlocking unit to bend accordingly, thereby controlling the rotation of the inspection module.

### BACKGROUND OF THE INVENTION

1. Field of the Invention

The present invention relates to an auxiliary structure for an endoscope, and in particular to a flexible tubular interlocking structure for a handheld endoscope.

2. Description of Prior Art

Various kinds of endoscopes are developed for inspecting the interior of a human body in a medical operation and for inspecting the interiors of machines for repair. Further, it is necessary to provide an auxiliary tool for the endo scope in order to improve the accuracy and precision of the endoscope in use.

Taiwan Patent No.M384635 discloses an endoscope. The front end of the endoscope is assembled with a flexible structure having a driving unit. The driving unit is constituted of a plurality of sleeves. Each of the sleeves is provided with square through-holes. Both sides of each sleeve are provided with a trough and a locking slot. Electrical wires pass through the through-holes, and a dragging portion pass through the troughs on both sides of the sleeve. The dragging portion is used to pull the driving unit, thereby controlling the driving unit to bend toward a desired direction. However, the through-holes and troughs of the sleeves are open, so that the dragging portion may deviate from the troughs to rub against the electrical wires when the driving unit is bent. As a result, the outer surface of the electrical wires having an insulation layer may suffer damage to generate a short circuit. Further, the dragging portion cannot be firmly positioned in the troughs of the sleeves. The endoscope is provided with a handle portion having a control unit. However, the control unit has to be operated by two hands, which makes the endoscope inconvenient in use. Therefore, it is an important issue for the present Inventor to solve the above-mentioned problems of the conventional endoscope.

### SUMMARY OF THE INVENTION

In order to solve the above problems, an objective of the present invention is to provide a flexible tubular interlocking structure for a handheld endoscope. The endoscope includes a handle portion and a flexible rod connected to one end of the handle portion. One end of the flexible rod is connected with an interlocking unit having an inspection module. The interlocking unit is provided with driving wires, and the handle portion is provided with a steering device. When a user pushes the handle portion to rotate the steering device, the driving wires inside the interlocking unit can be pulled to make the interlocking unit to bend leftward or rightward. In this way, the inspection module provided at the front end of the interlocking unit can perform the inspection in different directions.

### BRIEF DESCRIPTION OF THE DRAWINGS

FIG. 1 is a perspective view showing an interlocking structure for a handheld endoscope according to the present invention;

FIG. 2 is another perspective view showing the interlocking structure for a handheld endoscope according to the present invention;

FIG. 3 is a perspective view showing an interlocking unit of the interlocking structure for a handheld endoscope according to the present invention;

FIG. 4 is an exploded view showing the interlocking unit of the interlocking structure for a handheld endoscope according to the present invention;

FIG. 5 is a schematic view showing electrical wires and driving wires penetrate the interlocking unit of the interlocking structure for a handheld endoscope according to the present invention;

FIG. 6 is a schematic view showing the bending of the interlocking unit of the interlocking structure for a handheld endoscope according to the present invention;

FIG. 7 is a schematic view showing an inspection module of the interlocking structure for a handheld endoscope according to the present invention;

FIG. 8 is a schematic view showing another inspection module of the interlocking structure for a handheld endoscope according to the present invention; and

FIG. 9 is a schematic view showing that light is reflected by a cap of the inspection module of the interlocking structure for a handheld endoscope according to the present invention.

### DETAILED DESCRIPTION OF THE INVENTION

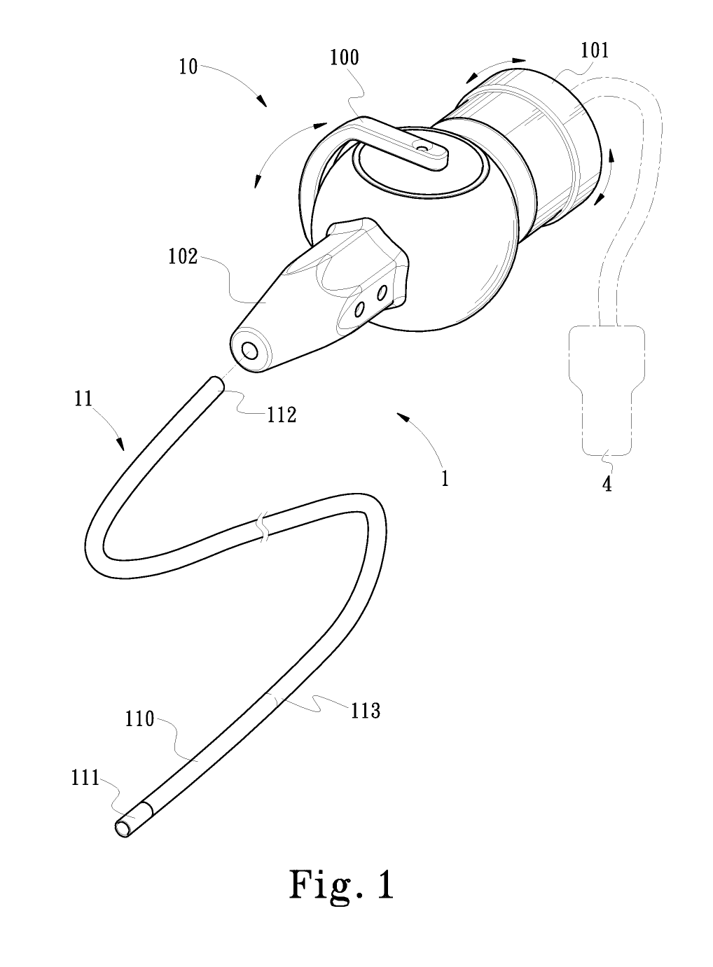

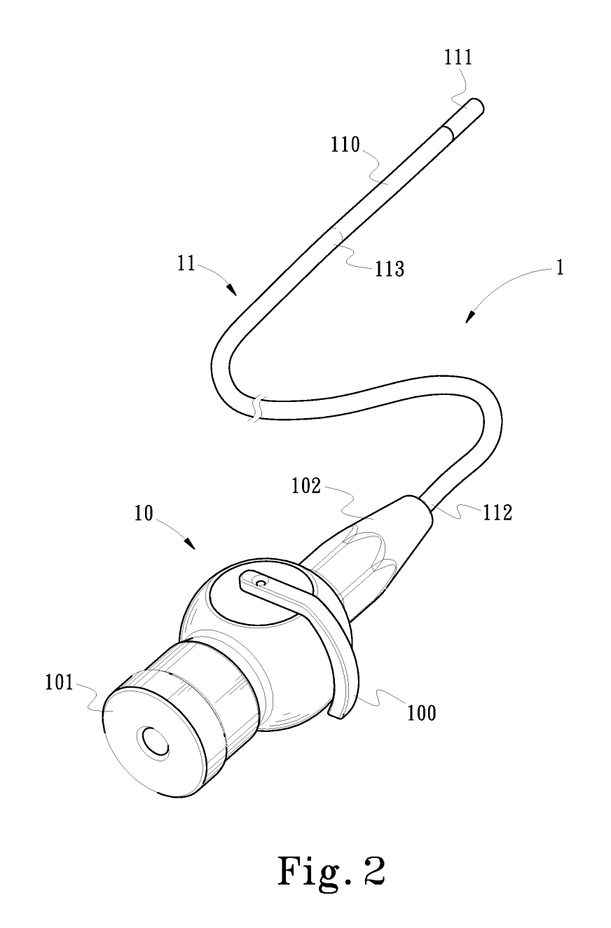

Please refer to FIGS. 1 and 2. The endoscope 1 of the present invention includes a handle portion 10 and a steering device 100 connected to a middle section of the handle portion 10. The rear end of the handle portion 10 is provided with a connecting ring 101, and the front end of the handle portion 10 is formed with a connecting pipe 102.

A flexible rod 11 has a first end 112 and a second end 113. The first end 112 is connected to the front end of the connecting pipe 102 of the handle portion 10 and in turn connected to the steering device 100. The second end 113 is connected to an interlocking unit 110. One end of the interlocking unit 110 is connected to a cap 111. The movement and rotation of the steering device 100 cause the interlocking unit 110 to rotate leftwards or rightwards. The rotation of the connecting ring 101 causes the flexible rod 11 to rotate accordingly. The connecting ring 101 is connected to a display device.

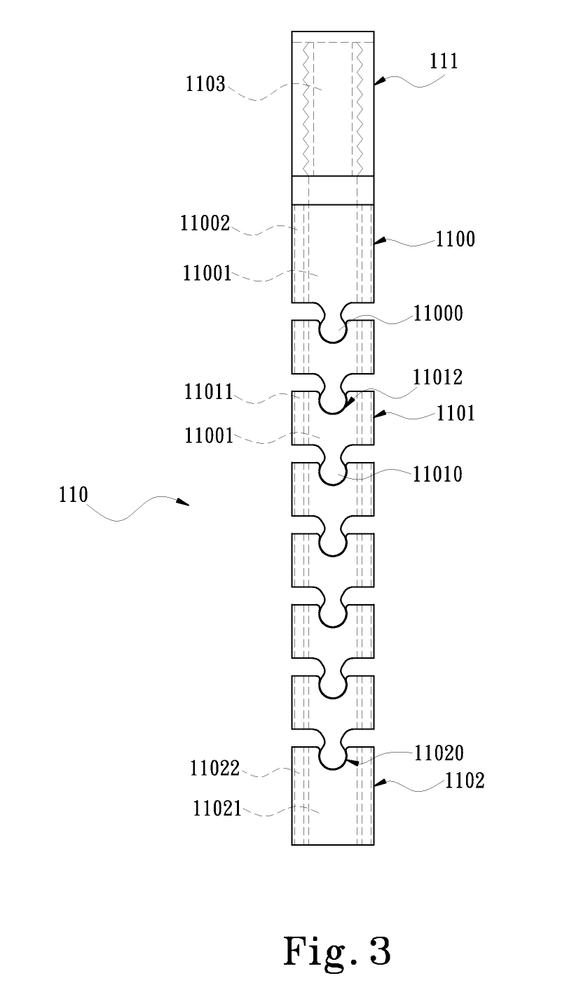

Please refer to FIGS. 3, 4, 5, and 6. The interlocking unit 110 comprises a front joint 1100, a plurality of middle joints 1101 and a rear joint 1102. The front joint 1100 is a tubular body having a hollow portion 11001. The outer periphery of one end of the front joint 1100 is formed with locking tabs 11000. The front joint 1100 is formed on its periphery with guiding holes 11002 in parallel to the axial line of the front joint 1100. The middle joint 1101 is a tubular body having a hollow portion 11013. The outer periphery of one end of the middle joint 1101 is formed with locking tabs 11010. The other end of the middle joint 1101 is provided with locking notches 11012. The middle joint 1101 is formed on its periphery with guiding holes 11011 in parallel to the axial line of the middle joint 1101. The rear joint 1102 is a tubular body having a hollow portion 11021. The outer periphery of one end of the rear joint 1102 is formed with locking notches 11021. The rear joint 1102 is formed on its periphery with guiding holes 11022 in parallel to the axial line of the rear joint 1102. The front joint 1100 and the rear joint 1102 are respectively connected to the front end and rear end of the middle joints 1101 to form the interlocking unit 110.

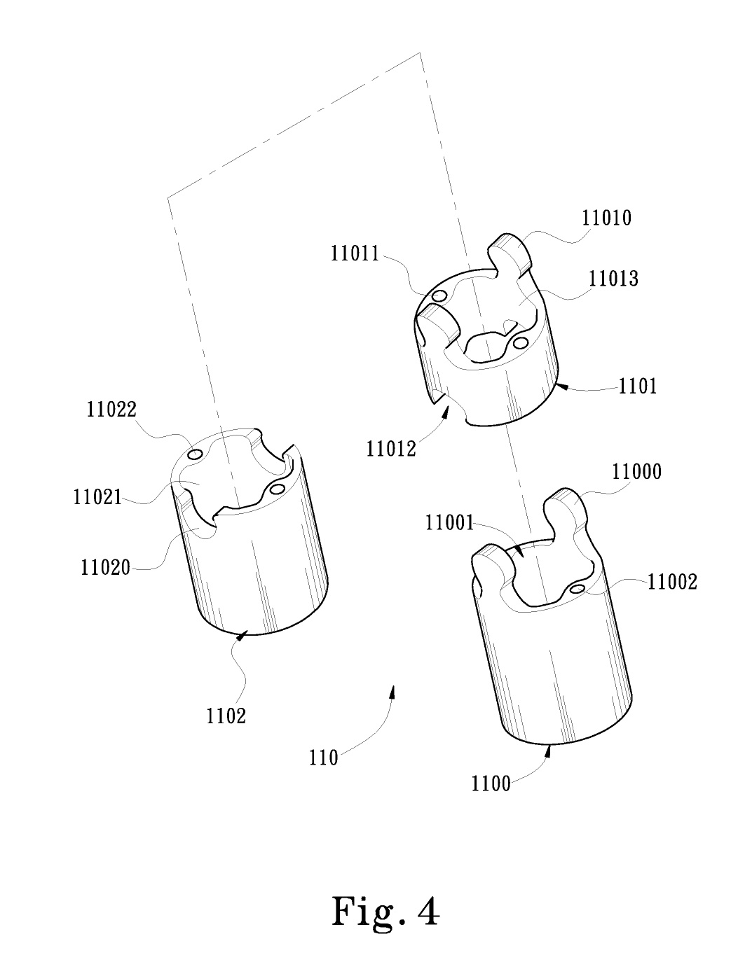

Please refer to FIG. 4 again. As mentioned in the above, the interlocking unit 110 is constituted of the front joint 1100, the middle joints 1101 and the rear joint 1102. The rear joint 1102 of the interlocking unit 110 is connected to the second end 113 of the flexible rod 11 (FIG. 1). The front joint 1100 is connected to an inspection module 1103. The cap 111 is threadedly connected to the outer periphery of the inspection module 1103 to protect the inspection module 1103.

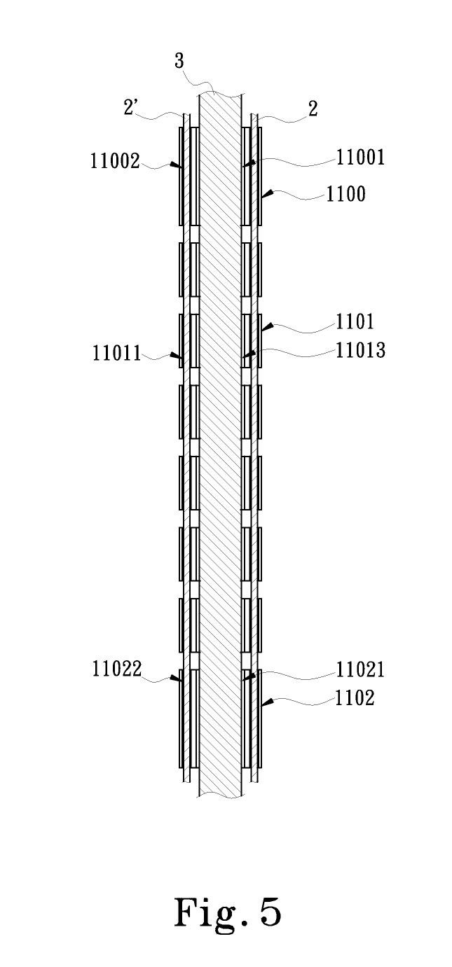

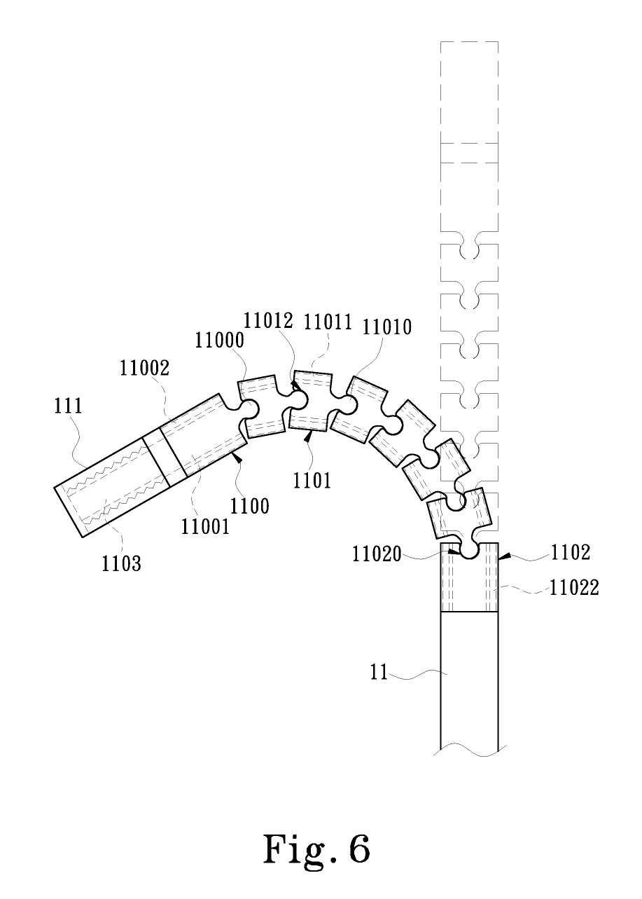

Please refer to FIGS. 5 and 6. As shown in FIG. 5, the locking tabs 11010 and the locking notches 11012 of the middle joints 1101 are connected to each other. Then, the locking notches 11020 of the rear joint 1102 are connected with the locking tabs 11010 of the middle joint 1101. The locking tabs 11000 of the front joint 1100 are connected with the locking notches 11012 of the middle joint 1101 (FIG. 4). After the front joint 1100, the middle joints 1101 and the rear joints 1102 are connected with each other in series, the guiding holes 11002, 11011, 11022 are located to correspond to each other, so that driving wires 2 and 2′ can be disposed through these guiding holes 11002, 11011 and 11022. A transmission cable 3 is disposed through the hollow portions 11021, 11013, and 11001.

As shown in FIG. 6, the driving wires 2 and 2′ are disposed through the front joint 1100, the middle joints 1101 and the rear joint 1102. One end of the driving wires 2 and 2′ are fixedly connected in the front joint 1100, and the other end thereof is fixedly connected to the steering device 100 of the handle portion 10 (FIG. 1). The upward or downward rotation of the steering device 100 tensions one of the driving wires 2, 2′ and loosens the other of the driving wires 2, 2′. Since the locking tabs 11000, 11010 and the locking notches 11012, 11020 of the front joint 1100, the middle joints 1101 and the rear joint 1102 are connected to each other by means of curved surface contact, so that the front joint 1100, the middle joints 1101 and the rear joint 1102 can be bent relative to each other. The rotation of the connecting ring 101 of the handle portion 10 causes the interlocking unit 110 to rotate in 360 degrees.

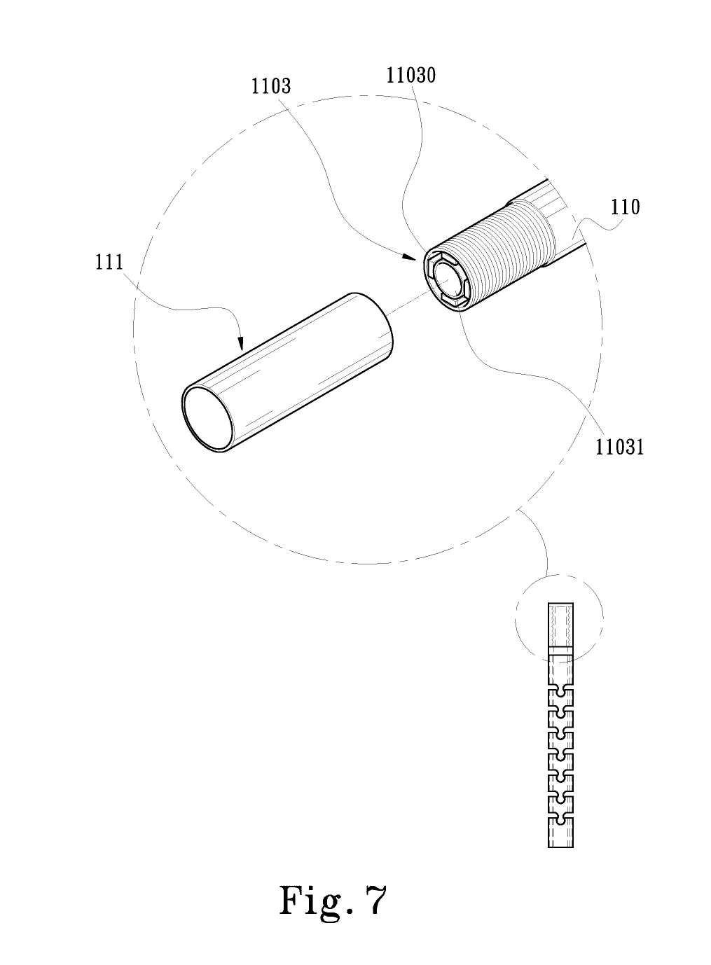

Please refer to FIGS. 7, 8 and 9. One end of the interlocking unit 110 is connected to the inspection module 1103. The inspection module 1103 includes a lens 11031 and a plurality of LEDs 11030. The LEDs 11030 are provided on the periphery of the lens 11031. The periphery of the inspection module 1103 is formed with outer threads, so that the hollow cap 111 can be threadedly connected to the inspection module 1103 for protection.

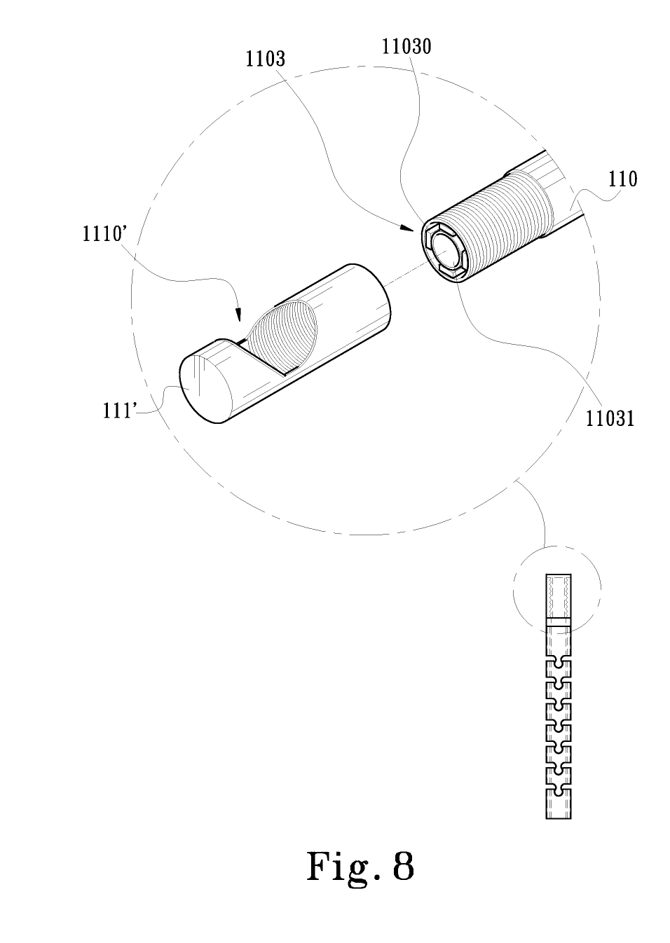

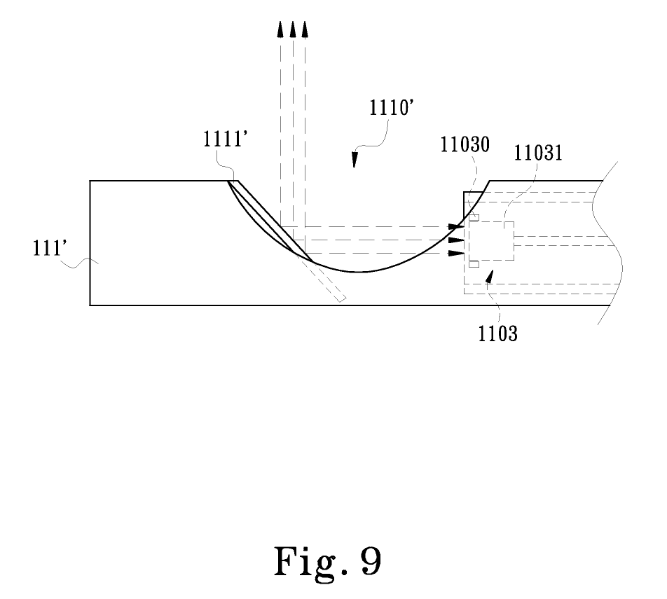

Please refer to FIGS. 8 and 9. The cap may be configured as another cap 111′ having a hollow end and an opening 1110′ on its outer periphery. The cap 111′ is threadedly connected to one end of the interlocking unit 110. A reflector 1111′ is provided in the opening 1110′ of the cap 111′. The reflector 1111′ reflects the light emitted by the inspection module 1103, so that the inspection module 1103 can take pictures and record images of objects to be inspected. The reflector 1111′ has an inclination angle in a range of 35 to 70 degrees, 45 to 90 degrees, and 55 to 110 degrees, so that the inspection can be performed in different directions. When the endoscope 1 is connected to a display device 4 as shown in FIG. 1, the display device 4 controls the inspection module 13 to take pictures and record images of objects to be inspected. By means of the caps 111, 111′, the steering device 100 and the connecting ring 101 shown in FIG. 1, the inspection can be performed in different directions, thereby inspecting the interiors of machines or small channels precisely.

What is claimed is:

1. A flexible tubular interlocking structure for a handheld endoscope, including:
a flexible rod configured as a tubular body having a first end and a second end, the second end being connected to an interlocking unit, the interlocking unit having a front joint, a plurality of middle joints and a rear joint, each of the joints having a hollow portion and guiding holes, the adjacent front joints, middle joints and rear joints having at least two locking tabs and locking notches corresponding to each other, the engagement between the locking tabs and the locking notches of the adjacent joints constituting the interlocking unit;
a handle portion having a connecting pipe connected to the first end of the flexible rod;
an inspection module connected to the second end of the interlocking unit;
at least one transmission cable and at least two driving wires, penetrating the flexible rod and the hollow portion and the guiding holes of the interlocking unit respectively, the transmission cable and the driving wires being connected between the handle portion and the inspection module;
wherein the driving wires are pulled to cause the interlocking unit to be bent leftwards or rightwards when the steering device of the handle portion is operated, the locking ring causes the interlocking unit to rotate in 360 degrees, thereby controlling the rotation of the inspection module.

2. The flexible tubular interlocking structure for a handheld endoscope according to claim 1, wherein one end of the inspection module is provided with a cap for protecting the inspection module.

3. The flexible tubular interlocking structure for a handheld endoscope according to claim 2, wherein one side of the outer surface is provided with an opening, a reflector is provided in the opening, so that the inspection of the inspection module can be performed in multiple directions.

4. The flexible tubular interlocking structure for a handheld endoscope according to claim 3, wherein the reflector is a magnifier.

5. The flexible tubular interlocking structure for a handheld endoscope according to claim 3, wherein the reflector has an inclination angle in a range of 35 to 70 degrees, 45 to 90 degrees, and 55 to 110 degrees.

6. The flexible tubular interlocking structure for a handheld endoscope according to claim 1, wherein the inspection module comprises a lens and a plurality of LEDs arranged on the periphery of the lens.

7. The flexible tubular interlocking structure for a handheld endoscope according to claim 1, wherein the handle portion comprises a steering device, a connecting ring and a connecting pipe.

8. The flexible tubular interlocking structure for a handheld endoscope according to claim 7, wherein the connecting ring of the handle portion is connected to a display device.
